(19) United States
(12) Patent Application Publication
(10) Pub. No.: US 2009/0220566 A1
(43) Pub. Date: Sep. 3, 2009

(54) BIOIMPLANTS FOR USE IN TISSUE GROWTH

(76) Inventors: Jake Barralet, Montreal (CA); Charles Doillon, Quebec (CA)

Correspondence Address: HOGAN & HARTSON LLP;IP GROUP, COLUMBIA SQUARE, 555 THIRTEENTH STREET, N.W., WASHINGTON, DC, 20004, US

(21) Appl. No.: 12/278,105

(22) PCT Filed: Feb. 1, 2007
(86) PCT No.: PCT/CA2007/000145

§ 371 Date: Apr. 10, 2009

Related U.S. Application Data
(60) Provisional application No. 60/763,930, filed on Feb. 1, 2006.

(51) Int. Cl. A61F 2/02

(52) U.S. Cl. 424/423

(57) ABSTRACT

Disclosed herein is an implant for use in stimulating tissue growth. The implant comprises a body with a body core and a body surface. The body is made from a non-hydrogel polymer material. A tissue growth stimulating material is disposed within the body core or deposited onto the body surface, in an amount which is sufficient to stimulate tissue growth within the body core or adjacent to the body surface. Also disclosed are implant bodies made from ceramic, metallic materials, and non-copper containing hydrogels.

### FIELD OF THE INVENTION

The present invention concerns bioimplants, and more particularly to bioimplants for use in promoting tissue growth or repair by stimulating tissue growth.

### BACKGROUND OF THE INVENTION

Blood vessel formation often precedes tissue healing, thus acceleration or induction of blood vessel formation can be beneficial for tissue repair. There are many materials used in bioimplants for tissue repair that may perform a structural, tissue guiding, mechanical, therapeutic, corrective, space filling, scaffolding, cosmetic, seeded or transplanted cell support, or delivery function, or any combination thereof. These materials are generally known in the field as biomaterials and tissue engineered devices or hybrid materials. These materials may be synthetic, naturally derived or combinations of both. Naturally derived materials are known as autografts, allografts, xenografts or may be animal or human or plant derived or recombinant analogues or cell derived.

Synthetic biomaterials are generally classed as metals, polymers, ceramics or composites thereof. Metals often have orthopaedic and dental applications and include stainless steel, titanium, tantalum. Polymeric biomaterials consist of two subclasses, namely polymers and hydrogels, the distinction mainly lying in hydrogels being swollen polymer networks containing significant (>50%) quantities of water, (more typically >85%). Examples of hydrogels include crosslinked alginates, non-fibrillar collagens, PEG (polyethylene glycol), PAA (polyacrylic acid), HEMA (hydroxy ethyl methacrylate). Polymers include PE (polyethylene), PGA (polyglycolic acid), PLA (poly lactic acid), PU (polyurathanes), PHB (polyhydroxybutyrate), and PTFE (polytetrafluoroethylene). Bioceramics include but are not limited to hydroxyapatite, calcium phosphate, calcium hydrogen phosphate, calcium carbonate, calcium silicates, zeolites, artificial apatite, brushite, calcite, gypsum, phosphate calcium ore, α and or β tricalcium phosphate, octacalcium phosphate, calcium pyrophosphate (anhydrous or hydrated), calcium polyphosphates (n≧3) dicalcium phosphate dehydrate or anhydrous, iron oxides, calcium carbonate, calcium sulphate, magnesium phosphate, calcium deficient apatites, amorphous calcium phosphates or crystalline or amorphous calcium carbonates or pyrophosphates or polyphosphates. Ceramics generally contain one or more of titanium, zinc, aluminium, zirconium, magnesium, potassium, calcium, iron, and sodium ions or atoms in addition to one or more of an oxide, a phosphate (ortho, pyro, tri, tetra, penta, meta, poly etc), a silicate, a carbonate, and a sulphate ions. Bioceramics further include composities thereof with metallic, ceramic and polymeric phases that can be used for example as bone or tooth replacement.

Examples of natural materials include but are not restricted to alginates, chitins, chitosans, tendon allograft, bone autograft, collagens and modified cellulose (for example, cellulose acetate). Natural materials may be combined with synthetic materials.

Often tissue integration is required and this is known to be enhanced by creating macroporosity (e.g. >200 μm) either at the bioimplant surface or throughout the bulk of the implant materials, and throughout the device. Furthermore, the bioimplant may be soluble and/or resorbable and/or degradable and/or hydrolysable to some degree.

Angiogenesis and vascularization represent the revascularization by microvessels or capillaries in tissues for blood supply and nutrient exchange, by the migration of cells such as endothelial cells. However, the rate of microvessel invasion can be slow after injury, grafting, or in healing-compromised patients, or biomaterial implantation, or tissue engineered implant (cell-containing implant) to reconnect the initial or newly formed tissue to the host tissue. One important issue is that for implants to be integrated a vascularisation is desirable. Researchers continue to develop different strategies to induce angiogenesis and vasculogenesis, using growth factors such as VEGF amongst others. These growth factors are unstable, may be immunogenic and biodegradable, and require considerable cost and may require preservation until use.

Copper has been shown to enhance angiogenesis and wound healing (Sen et al., 2002), (Rajalingam et al., 2005). Conversely, copper chelation by specific components such as tetrathiomolybdate and tetraethylenepentamine is has been applied as an anti-angiogenic treatment (Godman et al., 2005; Grzenkowicz-Wydra et al., 2004). Bioceramics immobilize, through adsorption and/or ion exchange, heavy metals, such as silver, copper or zinc. This property has been used to develop disinfectant bio-ceramic materials Atsumi et al., U.S. Pat. No. 5,151,122).

Previous studies demonstrate that copper could play an important role when incorporated in an implant that consists of a crosslinked glycosaminoglycan (hyaluronic acid) polymer (called Hyal-Cu) in order to enhance wound healing and angiogenesis (Barbucci et al., 2005; Giavaresi et al., 2005; Barbucci et al., U.S. Pat. No. 6,831,172). These reports relate specifically to quantities of copper greater than 75 μg of copper (not copper salt) per gram composition. Barbucci discloses implants comprising 0.21 mg copper sulfate in 1 g Hyal bulk material, which is equivalent to 80 μg copper per g composition, and 10 times this concentration was used to stimulate endothelial cells growth in vitro. Giavaresi's reference discloses in vivo implantation of a material comprising 24 μg copper ion per gram dry hydrogel. Since high amounts of copper may be toxic, it may be desirable to reduce the amount of copper exposed to biological tissues without compromising the pro-angiogenic effect that is sought for enhancing the colonization of endothelial cells into the implant.

Thus, there is a need for better, faster and more cost effective tissue repair strategies that can be carried out using a combination of bioactive components such as metals and their salts, and bioimplants.

### SUMMARY OF THE INVENTION

We have made the unexpected discovery that bioimplants made of non-hydrogel material containing a tissue growth stimulating amount of either a metallic or non-metallic material can cause localized tissue generation, such as vascularization, angiogenesis and microvessel formation, during transient or pulse-release of the metallic or non-metallic materials. Advantageously, the implants can be used to promote wound healing in patients. In addition, the implants reduce the need for growth factors The implants may also be easily stored before implantation.

Accordingly, there is provided in an embodiment of the present invention an implant for use in stimulating tissue growth, the implant comprising:
a) a body having a body core and a body surface, the body being made from a non-hydrogel polymer material; and
b) a tissue growth stimulating material being disposed within the body core or located on the body surface in an amount which is sufficient to stimulate tissue growth within the body core or adjacent to the body surface.

Accordingly, in another embodiment of the present invention there is provided an implant for use in stimulating tissue growth, the implant comprising:
a) a body having a body core and a body surface, the body being made from a ceramic material; and
b) a tissue growth stimulating material being disposed within the body core or located on the body surface in an amount which is sufficient to stimulate tissue growth within the body core or adjacent to the body surface.

Accordingly, in another embodiment of the present invention, there is provided an implant for use in stimulating tissue growth, the implant comprising:
a) a body having a body core and a body surface, the body being made from a metallic material; and
b) a tissue growth stimulating material being disposed within the body core or located on the body surface in an amount which is sufficient to stimulate tissue growth within the body core or adjacent to the body surface.

Accordingly, in another embodiment of the present invention, there is provided an implant for use in stimulating tissue growth, the implant comprising:
a) a body having a body core and a body surface, the body being made from a hydrogel polymer material; and
b) a tissue growth stimulating material being disposed within the body core or located on the body surface in an amount which is sufficient to stimulate tissue growth within the body core or adjacent to the body surface, the tissue growth stimulating material being selected from either a non-metallic material or a metallic material selected from cobalt, iron, zinc, magnesium or manganese.

Accordingly, in an alternative embodiment of the present invention, there is provided an implant for use in stimulating tissue growth, the implant comprising:
a) a body having a body core, and a first and second body openings, the body openings being in communication with the body surface;
b) a branched passageway extending between the body core and the body openings and in communication therewith, the branched passageway having a blind end portion; and
c) an amount of a tissue growth stimulating material being locatable near the blind end portion, the material being diffusible into the passageway and away from the body openings so as to stimulate tissue growth in the passageway, within the body core or adjacent to the body surface.

Accordingly, in another embodiment of the present invention, there is provided use of the implant, as described above, for stimulating tissue growth in a patient

Accordingly, in another embodiment of the present invention, there is provided use of the implant, as described above, for wound healing.

Accordingly, in another embodiment of the present invention, there is provided a method for stimulating tissue growth in a subject, the method comprising:
a) implanting the implant, as described above, at a location in the subject requiring such stimulation; and
b) comparing the amount of tissue growth in the implant or of the surface of the implant, or an area adjacent to the implant to that of a control, an increase in the amount being an indication that tissue growth has been stimulated.

In aspects of the aforesaid implants, tissue growth stimulating material is a metallic material. The metallic material includes an elemental metal, a metal ion, a metal-containing polypeptide, a metal-containing protein, a metal-binding protein, a metal-containing polymer, a metal-binding polymer, a metal complexing protein, or a metal complexing polymer. In one example, the elemental metal is copper. In another example, the elemental metal is cobalt. The metal-containing protein is ferroxidase (ceruloplasmin) or a copper-based hemocyanin. The metal-binding protein is albumin, alginate, or albumin PEG. Typically, the metal ion is present as a metal salt. The metal salt is selected from the group consisting of: copper sulfate, copper chloride, copper bromide, copper iodide, copper nitrate, copper nitrite, copper phosphate, copper phosphites, copper phosphides, copper pyrophosphates, copper polyphosphates, copper phosphonates, copper sulphites, copper sulphides, copper carbonates, copper oxides, copper silicates, copper salicylates, copper ascorbate, copper hydroxyacid salts (lactates, acetates, citrates), krebs acid salts of copper, copper oxalates, copper urates, cobalt sulfate, cobalt chloride, cobalt bromide, cobalt iodide, cobalt nitrate, cobalt phosphate, cobalt phosphites, cobalt phosphides, cobalt pyrophosphates, cobalt sulphites, cobalt sulphides, cobalt carbonates, cobalt oxides, cobalt silicates, cobalt salicylates, cobalt ascorbate, cobalt hydroxyacid salts (lactates, acetates, citrates), krebbs acid salts of cobalt, copper oxalates, cobalt urates, cobalt chloride, cobalt oxide, cobalt acetate, cobalt fluoride, cobalt oxide, cobalt phosphate, cobalt hydrate, cobalt sulfate, and cobalt selenite, cobalt polyphosphates, cobalt phosphonates, and cobalt phthalocyamine. In one example, the metal salt is copper sulfate. In another aspect of the aforesaid implants, the tissue growth stimulating material is a non-metallic material. In one example, the non-metallic material is elemental selenium. In another example, the non-metallic material is a selenium salt. Typically, the selenium salt is selected from the group consisting of: ammonium selenide, ammonium selenate, ammonium selenite, selenium hydride, sodium selenite, potassium selenite, magnesium selenite, lithium selenite, beryllium selenite, potassium selenite, calcium selenite, selenium chloride, selenium bromide, selenium oxide, selenium iodide, selenium fluoride, cobalt selenite, copper selenite or mixed salts thereof. In one example, the selenium salt is sodium selenite. Typically, the metal salt is soluble or sparingly soluble in water at a concentration of >10 μg/litre at 37° C. The implant, as described above, further comprising a supplementary metallic material suitable to stimulate tissue growth. Typically, the supplementary metallic material is Fe, Zn, Mg, Mn, or any combinations thereof. The implant, as described above, further comprising a vascular endothelial cell growth factor. The implant, as described above, further including a bone inducing factor. The implant, as described above, further including a growth factor. In one example, the vascular endothelial cell growth factor is VEGF or basic-FGF (b-FGF or FGF-2) or a combination thereof. In one example, the bone inducing factor is BMP. In one example, the growth factor is TGF-β. In one example of the aforesaid implants, the metallic material is a copper ion, the copper ion being at a concentration of less than 20 μg copper ion per mm³ of implant material. In another example, the metallic material is a copper ion, the copper ion being at a concentration of less than 1 μg copper ion per mm³ of implant material. In another example, the copper ion is 0.1 ng copper ion per mm³ implant material or more and the copper ion is 3 μg copper ion/mm³ implant material or less. In one example, the metal salt is cobalt chloride. The cobalt chloride is at a concentration 0.45 ng per mm³ of the implant. In another example, the selenium salt is sodium selenite. The sodium selenite is at a concentration of 0.25 ng/mm³ of the implant. In one example, the ceramic material is brushite or hydroxyapatite. In one aspect. he amount of the tissue growth stimulating material is sufficient to stimulate angiogenesis. In another aspect, the amount of the tissue growth stimulating material is sufficient to promote vascularization. In another aspect, he amount of the tissue growth stimulating material is sufficient to promote microvessel formation. In another aspect, the body surface is colonizable by vascular endothelial cells. In one example, the non-hydrogel polymer material is a synthetic non-hydrogel polymer or a natural non-hydrogel polymer. The tissue growth stimulating material is transiently released from the body core or the body surface. The tissue growth stimulating material is pulse released from the body core or the body surface.

In an example of the aforesaid implant, the body includes at least two mateable body portions. Each body portion includes a complementary body channel, the body channels, when the body portions are mated, form the branched passageway. A first body portion includes a pair of projections and a second body portion includes a pair of recesses, the projections being sized and shaped to engage the recesses. The branched passageway is Y-shaped. The implant is a cuboid.

### BRIEF DESCRIPTION OF THE FIGURES

Further aspects and advantages of the present invention will become better understood with reference to the description in association with the following Figures, wherein:

FIG. 1A is a photograph plan view of two halves of an embodiment of an opened implant of the present invention.

FIG. 1B is a perspective view of an implant showing the location of two body openings.

FIGS. 1C and 1D is a micro-computed tomography showing the position of pores within the implant.

FIG. 2 is an series of photographs illustrating a copper-impregnated pore structure in a bioceramic implant (brushite implants with copper). Y-shaped channel structures were made in a two-halved implant as represented in photograph A (24: main pore; 22: open pore; and 26: blind closed pore). 56 ng CuSO₄ was adsorbed on the blind closed pore as represented in B (blue area). 15 days after peritoneal implantation in mice, the halves were opened to observe the tissue ingrowth (photograph C represents the copper-impregnated material and photograph D the control implant without copper). Newly formed microvessels were observed particularly in the main (C1) and blind closed pores (C3) in the presence of copper. Microscopic observation of the new tissue confirmed the presence of microvessels (Cm). Photographs D1 to Dm are of control implants show limited vascularization into open pores (D1 and D2) with none in the blind closed pore (D3), as seen by microscopic observation (Dm).

FIG. 3 is a graph shows the mean distance over which blood vessels were observed to grow from the large pore opening in FIG. 2A to the closed pore end. The dotted line shows the total distance from the pore opening to the closed end. Both copper and VEGF increased blood vessel in-growth from 2 mm in the control, to nearly the entire length for implants treated with 56 ng of copper sulphate at the pore end, 2 μg VEGF, and 200 ng VEGF and 56 ng copper sulphate combined after 15 days interperitoneal implantation. Lower quantities of VEGF or higher concentrations of copper sulphate alone resulted in a lesser mean blood vessel in-growth distance (3.7 and 4.3 mm respectively FIG. 4 is a photograph of a cobalt-loaded implant, in which the wound tissue was inflammatory, and by histology, microvessels with transgression of inflammatory cells were observed.

FIG. 5 is a photograph of a selenium-loaded implant, the tissue ingrowth was oriented towards the closed pore and histological sections showed an immature wound tissue.

FIG. 6 is a photograph of a peritoneal control implant, tissue filled with blood was found. Microscope examination showed no microvessel existing in the tissue extracted from the tube.

FIG. 7 is a photograph of the interior of the copper-coated tubes, a relatively extended wound tissue was observed.

### DETAILED DESCRIPTION OF THE INVENTION

### Definitions

Unless stated otherwise, the following definitions apply:

The singular forms “a”, “an” and “the” include corresponding plural references unless the context clearly dictates otherwise.

As used herein, the term “comprising” is intended to mean that the list of elements following the word “comprising” are required or mandatory but that other elements are optional and may or may not be present.

As used herein, the term “consisting of” is intended to mean including and limited to whatever follows the phrase “consisting of”. Thus the phrase “consisting of” indicates that the listed elements are required or mandatory and that no other elements may be present. Don't you mean that other elements may be present?

As used herein, the term “cell” is intended to mean a single-cellular organism, a cell from a multi-cellular organism or it may be a cell contained in a multi-cellular organism, or a plurality of non-interconnected cells Tissue, as used herein is intended to mean a collection of interconnected cells that perform a similar function within the a subject.

As used herein, the term “subject” or “patient” is intended to mean humans and non-human mammals such as primates, cats, dogs, swine, cattle, sheep, goats, horses, rabbits, rats, mice and the like. In one example, the subject is a human.

As used herein, the term “protein”, “polypeptide” or “polypeptide fragment” is intended to mean any chain of two or more amino acids, regardless of post-translational modification, for example, glycosylation or phosphorylation, constituting all or part of a naturally occurring polypeptide or peptide, or constituting a non-naturally occurring polypeptide or peptide.

As used herein, the term “metal salt” is intended to include “acid addition salt” and “base addition salt” as defined below.

The term “acid addition salt” is intended to mean those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Examples of such salts include, but are not limited to phosphates, phosphites, phosphides, pyrophosphates, phosphonates, polyphosphates, chlorides, bromides, iodides, sulphates, sulphites, sulphides, carbonates, oxides, silicates, salicylates, ascorbates, hydroxyacid salts (such as lactates, acetates, citrates), krebbs acid salts, oxalates, and urates.

The term “base addition salt” is intended to mean those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like.

Examples of the aforesaid salts include, but are not limited to, phosphates phosphites, phosphides, pyrophosphates, phosphonates, polyphosphates, chlorides, bromides, iodides, sulphates, sulphites, sulphides, carbonates, oxides, silicates, salicylates, ascorbate, hydroxyacid salts (such as lactates, acetates, citrates), krebbs acid salts, oxalates, and urates.

Examples of selenium salts, include, but are not limited to, ammonium selenide, ammonium selenate, ammonium selenite, selenium hydride, sodium selenite, potassium selenite magnesium selenite, lithium selenite, beryllium selenite, potassium selenite, calcium selenite, selenium chloride, selenium bromide, selenium oxide, and selenium iodide, cobalt selenite, and copper selenite.

Another example of a salt or an element includes, either singly or in combination, copper, cobalt and selenium.

As used herein, the term “non-hydrogel polymer” is intended to mean polyurethane, polyester, polytetrafluoroethylene, polyethylene, polymethylmethacrylate, polysiloxanes, and all poly hydroxyacids. Examples of non-hydrogel polymers include, but are not limited to the following:

| Synthetic polymers | Natural polymers |
|---|---|
| Poly lactic acid | Fibrin |
| Poly-L-lactic acid | Albumin |
| Poly-D,L-lactic acid | Casein |
| Poly glycolic acid* | Keratin |
| Poly-e-caprolactone | Fibrillar Collagen |
| Poly-p-dioxanon | silk fibroin |
| Tri-methylen carbonate | lipids |
| Poly anhydrides | phospholipids |
| Poly ortho ester | Amphiphiles |
| Poly urethanes | |
| Poly amino acids | |
| Poly hydroxy alcanoates | |
| Poly phosphazenes | |
| Poly-b-malein acid | |
| Polyhydroxybutyrate | |
| Polystyrenes e.g. Poly(styrene-co- | |
| chloromethylsytrene) | |
| lipids (e.g. monoolein) | |
| phospholipids | |
| Polyphosphoesters | |
| Polyphosphazenes | |
| Aliphatic Polyesters e.g. PCL PGA | |
| PLA & Copolymers | |
| PHB PHV & Copolymers | |
| Poly(1,4-butylene succinate), | |
| Nylons | |
| Non Hydrogel Polysaccharides, e.g. | |
| cellulose acetates | |
| PEG Based Polymers | |
| Poly(ethylene oxide) average | |
| Polyanhydrides | |
| Poly(butylene Terephthalate) | |
| Amphiphiles | |

As used herein, the term “implant” and “bioimplant’ are used interchangeably and is intended to mean the apparatus of the present invention, which support the tissue growth stimulating material and therefore promotes localized cell or tissue growth.

As used herein the term “tissue growth stimulating material” is intended to mean a material which induces tissue formation and/or differentiation and/or migration and/or proliferation.

As used herein, the term “stimulating tissue growth” is intended to mean causing an increase in growth of cells or tissues, and facilitating wound tissue infiltration. For example, causing increased angiogenesis, vasculogenesis, vascularisation or microvessel formation compared to tissue with an implant that has no tissue growth stimulating material implanted in the same site, in the same species, at the same time.

As used herein the term angiogenesis is intended to mean the growth of new blood vessels from preexisting blood vessels.

As used herein, the term “vasculogenesis” is intended to mean blood vessel formation from the de novo production of endothelial cells. Vasculogenesis occurs when endothelial precursor cells migrate and differentiate in the presence of adult endothelial cells to form new blood vessels in the adult. Circulating endothelial precursor cells (derivatives of stem cells) contribute, albeit to varying degrees, to neovascularization, or to the revascularization process following trauma, e.g. after cardiac ischemia.

As used herein, the term “ceramic” or “bioceramic” is intended to include all ceramics which may be formed from oxides, carbonates, carbides, nitrides, titanates, zirconates, silicates, phosphonates, phosphates, pyroposphates, polyphosphates, sulphides, sulphates, selenides, selanates, selenites, of calcium, sodium, potassium, aluminium, magnesium, zinc, silicon, strontium, barium, or transition metals.

As used herein, the term “disposed within” when used in connection with the tissue growth stimulating material, is intended to mean a material such as ion(s) and/or element(s) contained within a microcapsule, liposome, microbeads and the like or on their surfaces; any controlled and/or sustained release matrix that is dispersed throughout the implant whether it be a metal, ceramic or polymer or composite thereof. In the case of ceramics, where ceramic means a non-metallic inorganic material including carbon and carbides and nitrides or an oxide, including oxide, carbide and nitride layers on metals, or plasma or solution deposited coating on metal (e.g. for improved osteoconductivity or bone bonding) and also cements. The material may also be substituted or present in the crystal lattice, (e.g. minor impurity), mixed as a separate phase, e.g. copper phosphate grains or inclusions in a ceramic matrix, copper powder, fibers and the like, or present at grain boundaries.

Adsorbed on surface or located onto the surface of the implant body includes plasma deposited, vapour deposited, plated, ion implanted. The adsorption or disposition onto or into the body surface or body core may include chemical bonds such as ionic bonding, chelation, covalent bonds, hydrogen bonds, van de Waals bonding, or the material may be substituted in the body core or onto the body surface. The material can be bound in anyway, either chemically or physically to an adsorbed or otherwise incorporated molecule. For example, copper bound to albumin dispersed throughout a ceramic phase for example in pores

The material may also be mixed with a cement, mechanically alloyed, including grit blasting, sputtering and the like. In the case of polymers, the term “disposed within” is intended to mean bound in anyway, chemically or physically to an adsorbed or otherwise incorporated molecule. For example, copper bound to albumin dispersed throughout ceramic phase, for example in pores. The term also means mixed with a polymerizing or crosslinking polymer system, or mixed as separate phase, e.g. copper phosphate grains or inclusions in a ceramic matrix, copper powder, fibres and the like. The adsorption or disposition onto or into the body surface or body core may include chemical bonds such as ionic bonding, chelation, covalent bonds, hydrogen bonds, van de Waals bonding. The material may be plasma deposited, vapour deposited, plated, or ion implanted. In the case of metal and ceramic, the term “disposed within” is intended to mean substituted or present in the crystal lattice, (e.g. minor impurity), mixed as a separate phase, e.g. copper phosphate grains or inclusions in a ceramic matrix, copper powder, fibres and the like and present at grain boundaries. The adsorption or disposition onto or into the body surface or body core may include chemical bonds such as ionic bonding, chelation, covalent bonds, hydrogen bonds, van de Waals bonding. The material may be plasma deposited, vapour deposited, plated, or ion implanted. The material can be bound in anyway, chemically or physically to an adsorbed or otherwise incorporated into molecule. For example, copper bound to albumin dispersed throughout ceramic phase, for example in pores. Also included is any form of alloying, including mechanically alloying, grit blasting and the like, or sputtering. Disposed within may also include incorporation into an oxide or other non-metallic surface layer on a metal. Examples of metals useful in formation of the implant body core include magnesium and alloys, any of the transition metals and their alloys, such as for example, nitinol, titanium and titanium alloy, stainless steel, cobalt-chrome alloys, tantalum.

Also included within the definition of “disposed within” are any reservoirs in the aforesaid materials, which are designed to release metal and non-metallic materials, such as for example, capsules, channels, voids, pores, and the like. Once implanted, the implant may be biodegradable, such as through the action of enzymes, or it may hydrolyse, or it may be phagocytosed, or it may corrode, or it may remain undegraded in situ.

It should also be noted that any of the above materials need not be homogeneously distributed throughout the body core or located on the body or pore surfaces.

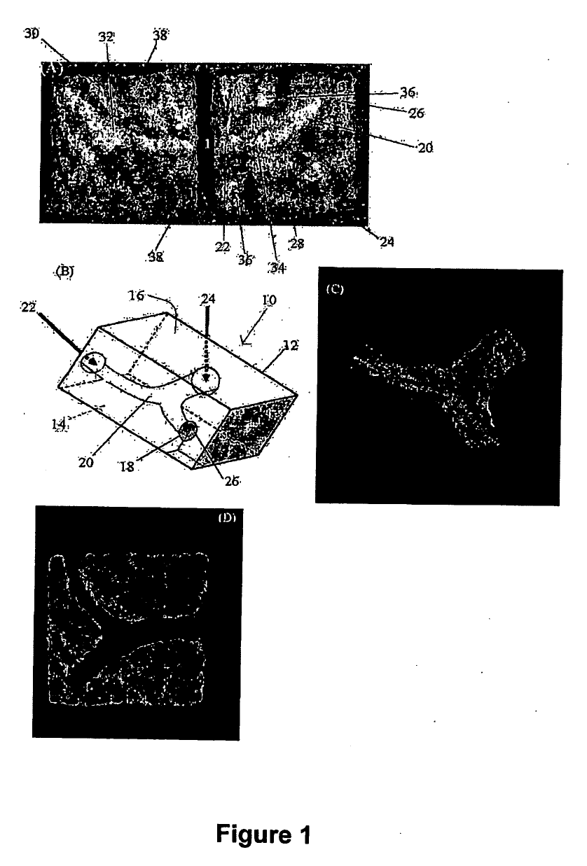

The present invention concerns bioimplants which are useful for in vivo efficiently inducing vasculogenesis, microvessel formation and angiogenesis early in the wound healing process. Referring now to FIGS. 1A-1D, an embodiment of an implant (or bioimplant) for stimulating tissue or cell growth according to the present invention is illustrated generally at 10. Broadly speaking, the implant 10 comprises a body 12 having a body core portion 14 and a body surface 16. A tissue growth stimulating material 18, which will be described in more detail below, is disposed either within the body core 14 or is deposited onto the body surface 16. The tissue growth stimulating material 18 is in an amount which is sufficient to stimulate tissue growth within the body core 14 or adjacent to the body surface 16. The tissue growth stimulating material 18 is typically diffusible throughout the passageway and away from the body openings.

In the embodiment shown in FIGS. 1A and 1B, the body 12 comprises a branched passageway 20 with a blind end and a body opening 22, with a second body opening 24 spaced apart from the first body opening 22 (also known as pores) which are in communication with the body surface 16 and the passageway 20. The passageway 20 extends between the body openings 22, 24. The passageway 20 has a blind end portion 26 located at one end of the passageway 20. In one example, the tissue growth stimulating material 18 is located at the blind end portion 26.

The body 12 includes first and second mateable body portions (or halves) 28, 30, which each includes complementary branched body channels 32, 34. The body channels 32, 34, when the body portions 28, 30 are mated together, form the branched passageway 20. In the example shown, the branched passageway 20 is Y-shaped.

The first body portion 28 includes a pair of projections 36 and the second body portion 30 includes a pair of recesses 38. The projections 36 are sized and shaped to lockingly engage the recesses 38 during mating of the two halves. Once mated, the body portions 26, 28 provide a body which is generally cuboid with the two body openings 22, 24 being disposed on two different cuboid surfaces. After use, the body portions 28, 30 may be disengaged to examine vascularization and tissue growth either within the passageway.

One skilled in the art will recognize that the body of the implant may be any number of shapes or may be an amorphous body. In the example shown, the cuboid dimensions are typically 8 mm×8 mm×3 mm.

In operation, the implant that can be disassembled to reveal contents of pores or channels post implantation or post cell migration. Soft tissues can be retrieved or examined without resin embedding and sectioning. The mateable body portions allow easy assembly by a push-fit, which reduce rotation and disassembly once implanted. The implant may be sutured close at the implantation site.

The invention provides an implant for use in stimulating tissue growth, in which the implant comprises a body having a body core and a body surface, the body being made from either a non-hydrogel polymer material, metal or a ceramic material. The tissue growth stimulating material is disposed within the body core or located on the body surface or located on the internal surface of the pores, (the pore maybe include macro, micro or nano porosity) in an amount which is sufficient to stimulate tissue growth within the body core or adjacent to the body surface. It is also within the scope of the present invention that the growth stimulating material 18 can be locatable onto the body surface 16 or disposable into the body core 14 immediately before implantation.

In one example, the tissue growth stimulating material is a metallic material, which includes an elemental metal, a metal ion, a metal-containing polypeptide, a metal-containing protein or a metal-binding protein, a metal-containing polymer, such as polyacrylic acid (PAA) and the like, or a metal-binding polymer. In one example, the elemental metal is either copper or cobalt or a combination of both.

In another example, the metal ion is a metal salt at least one of which is which is selected from the group consisting of: copper sulfate, copper chloride, copper bromide, copper iodide, copper fluoride, copper nitrate, copper phosphate, copper phosphites, copper phosphides, copper pyrophosphates, copper polyphosphates, copper sulphites, copper sulphides, copper carbonates, copper oxides, copper silicates, copper salicylates, copper ascorbates, copper hydroxyacid salts (lactates, acetates, citrates etc), krebs acid salts of copper, copper oxalates, copper urates, cobalt sulfate, cobalt chloride, cobalt bromide, cobalt iodide, cobalt nitrate, cobalt phosphate, cobalt phosphites, cobalt phosphides, cobalt pyrophosphates, copper selenite, cobalt polyphosphates cobalt sulphites, cobalt sulphides, cobalt carbonates, cobalt oxides, cobalt silicates, cobalt salicylates, cobalt ascorbate, cobalt hydroxyacid salts (lactates, acetates, citrates etc), krebbs acid salts of cobalt, copper oxalates, cobalt urates, cobalt chloride, cobalt oxide, cobalt acetate, cobalt fluoride, cobalt oxide, cobalt phosphate, cobalt hydrate, cobalt sulfate, cobalt selenite and cobalt phthalocyamine.

In a specific example, the metal salt is either copper sulfate or cobalt chloride.

In another example, the tissue growth stimulating material is a non-metallic material, which includes elemental selenium or a selenium salt. At least one selenium salt is selected from the group consisting of: ammonium selenide, ammonium selenate, ammonium selenite, selenium hydride, sodium selenite, potassium selenite, magnesium selenite, lithium selenite, beryllium selenite, potassium selenite, calcium selenite, selenium chloride, selenium bromide, selenium oxide, selenium iodide, selenium fluoride, cobalt selenite, copper selenite or mixed salts of the above, such as potassium sodium selenide.

In a specific example, the selenium salt is sodium selenite.

Generally speaking, the metal salts described above are soluble or sparingly soluble in aqueous media such as water or body fluids at ≧10 μg/litre at 37° C.

By a variety of art-recognized techniques it is possible to introduce metals, metal ions (salts and such), and non-metallic materials into the body of the implant or onto the surface of the implant.

We have demonstrated using calcium phosphate cements that concentrations of copper, which are below 20 μg/mm³ are suitable to encourage endothelial cell growth into a 1.3 mm diameter pore opening at the surface of an implant. In one example, the copper is located at least on the body surface 16 onto which endothelial cells colonization is sought. Typically, the copper concentration is less than 20 μg/mm³ of implant material, (in the case of an absorbant material such as a micro and/or nano porous ceramic) In another example, the copper concentration is less than 10 μg/mm³ of implant material In another example, the copper concentration is less than 1 μg/mm³ of implant material In a typical example, the concentration of copper is 0.1 ng/mm³ of implant material or more and 3 μg/mm³ implant composition or less. In the case of non-absorbent materials such as dense ceramic monoliths, non micro or mesoporous metals an equivalent amount may be deposited per mm².

The copper source for use in the implants may vary: Typically, copper salts like copper sulfate, copper sulfate pentahydrate, copper pyrophosphate, or copper nitrate, and the like, may be used.

The copper may be added to a metal by preparing both metals together, by adding copper ions on the metal surface, by implanting copper ions in the metal surface, by making composites or alloys of metal and copper, or by making copper alloyed with a metal surface.

The copper compound may be introduced to the ceramic by different ways, including: copper salts can be chemically substituted into the ceramic, they can be impregnated into the ceramic, copper salts can be coated onto the ceramic by diverse techniques, such as plasma coating or simple application of a copper solution and dried. Elemental copper may also be included (<20 μg per mm³ of implant). To induce both bone formation and angiogenesis, the addition of supplementary metal salts such as Fe, Zn, Mg, or Mn to copper may be beneficial.

In addition to copper, a protein or a combination of proteins such as growth factors (e.g., VEGF, bFGF) or bone inducing factors (BMPs, TGF-β) or extracellular component or bioactive (poly)peptide or protein(s) or combination thereof may be added to enhance the tissue response (i.e., newly formed vascularized bone tissue). Copper can be also combined with other elements such as Zn, Ca, and phosphates, described above, to substitute into the crystal lattice(s).

Metal-containing proteins such as, for example, ferroxidase (ceruloplasmin) or copper based hemocyanin are useful in the practice of the present invention. Similarly, metal-binding proteins such as, for example, albumin, alginate, or albumin PEG are useful.

Metal complexing proteins, or metal complexing or binding polymers are also useful in the practice of the present invention.

In addition, peptides and polypeptides (e.g., tripeptide- and tetrapeptide-copper complexes) are useful in the practice of the invention.

The material of the implant body may be of diverse types. For example, gels, polymers, metallic materials, ceramics, and composites thereof. In one example, the implant will comprise a ceramic component to provide the best matrix as possible for the reconstruction of bone tissue. In one example, the implant may be osteoconductive to facilitate the construction of bone tissue.

In another example, the implant is made from a metallic material.

In another example, the implant may be made from a hydrogel polymer. In a specific example, in this case, the hydrogel polymer can be used with either a non-metallic material selected from selenium or a metallic material selected from, cobalt iron, zinc, magnesium or manganese as the tissue growth stimulator.

In another example, the implant may include copper hydrogels disposed in the body of the implant or mixed as a composite. According to another example, a hydrogel may also be disposed within a copper containing implant.

The ceramics may also be of different types: for example, they may be sintered ceramics and ceramic composites with polymers, composites with metallic, ceramic and polymeric phases such as mineralized hydrogels, cements, and polymer beads. The ceramic can be a component in a composite with metal and copper, or it can be a component in a composite with polymer and copper, or it can be a component in a composite with ceramic and copper. Copper can be introduced during the manufacturing of the implants.

In general, any implant can comprise an effective amount of copper located onto a relevant colonizable surface by any suitable means, depending upon the nature and composition of the material which supports the same (plastic, metallic material, non-metallic material, hydrogel polymer, polymer, non-hydrogel polymer, and ceramic. The implant of the present invention may be made of any of the aforesaid materials or any composites thereof.

The implants can be used as, for example, bone or tooth replacement implants, which are implantable during surgery. They can also be designed with specific guidance patterns or macroporosity to orient tissue ingrowth upon implantation so as to promote vascularization, angiogenesis, or microvessel formation. The addition of growth factors that stimulates angiogenesis (newly formed vascularisation) may be useful, but a simple method has been developed to enhance angiogenesis in those materials.

Generally speaking, tissue growth can be in a human patient by implanting the implant of the present invention at a location in the patient's body that requires such stimulation, such as after bone trauma or in situations requiring enhanced bone healing, surgery, healing in compromised patients, such as in diabetics, or radiation-treated patients and the like. In addition, the implant may also be useful for soft tissue attachment to bone

The amount of tissue growth in the implant or of the surface of the implant, or an area adjacent to the implant can be compared to that of a control. An increase in the amount of angiogenesis, vascularization or microvessel formation indicates that tissue growth has been stimulated. In addition to angiogenesis, vascularization or microvessel formation; tissue differentiation, migration, remodeling or lack of fibrous tissue formation may also be analyzed and compared to the control

Generally speaking, the amount of the tissue growth stimulating material is sufficient to cause an angiogenic response with or without an minor inflammatory response. An adverse response would be inflammation without blood vessel formation or a significant cytotoxic effect and or chronic inflammation and or necrosis.

According to an alternative embodiment, the invention provides a bioimplant comprising an angiogenic amount of a copper ion exposed at least at a surface colonizable by vascular endothelial cells, this amount being less than 20 micrograms per mm³ in any portion of the implant material.

According to an alternative embodiment, the invention provides a bioimplant comprising an angiogenic amount of a copper ion exposed at least at a surface colonizable by vascular endothelial cells, the amount being less than 70 micrograms per mm³ in any cm³ of implant material.

### EXAMPLES

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

Animal models which are used in the following Examples have been validated as models for use of implants in human patients (see for example ISO 10993 series as exemplified in: An MD&DI August 1998 Column: A Practical Guide to ISO 10993-6: Implant Effects; R. F. Wallin and P. J. Upman; Metals and Materials; Opolka A, et al in Matrix Biol. 2006 Oct. 3; Duvall C L, et al in J Bone Miner Res. 2007 February; 22(2):286-97; Colnot C, et al in Biochem Biophys Res Commun. 2006 Nov. 24; 350(3):557-61; Lu C, et al in J Orthop Res. 2007 January; 25(1):51-61; Egermann M, et al in Osteoporos Int. 2005 March; 16 Suppl 2:S129-38; and Manigrasso MB, and O'Connor JP in J Orthop Trauma. 2004 November-December; 18(10):687-95).

### Example 1

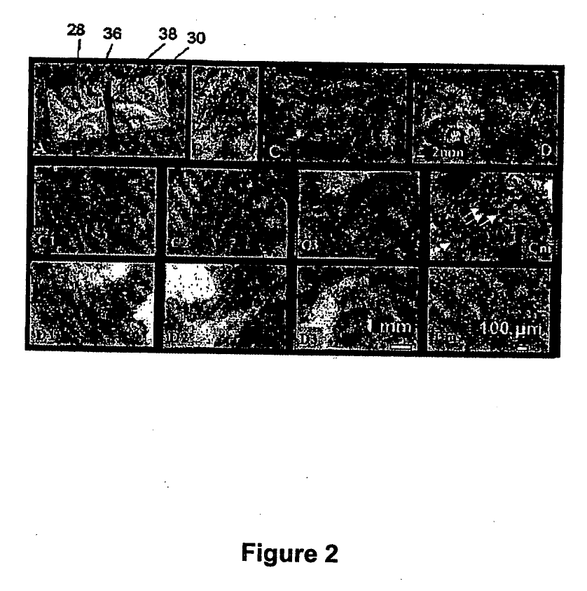

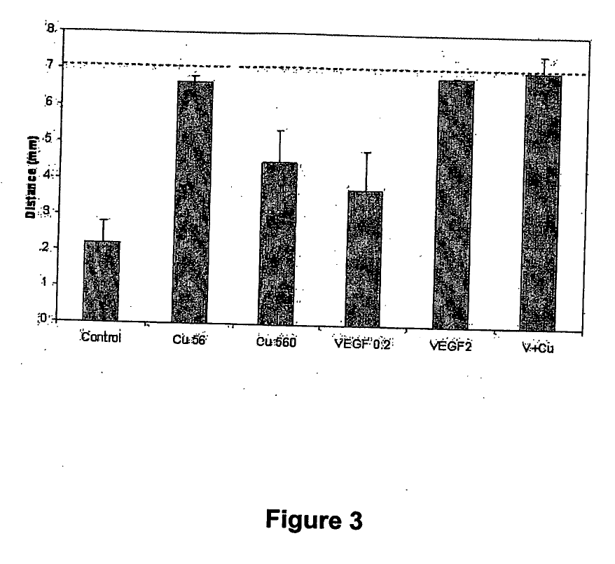

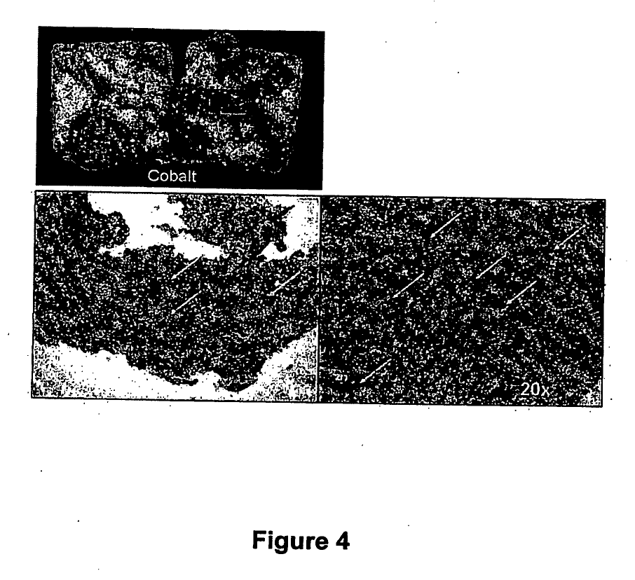

Brushite and hydroxyapatite cuboid bioimplants (434±4 mg dry weight) with a 2D branched structure (Y-shape pore) were produced by cement printing following a method previously developed (Hölzel, 2005). The pore was 1.31 mm diameter and open in the middle of one smaller face and decreased in diameter to 1 mm as it branched in the centre of the block. One branch emerged on an adjacent face while the other was a ‘blind’ closed pore. 5 μl of 70 μM copper sulphate solution was deposited at the end of the blind closed pore of the Y as represented in FIG. 2B. This solution thus contained a total of 350 pM of copper sulphate, i.e. 22 ng of copper ions. It was absorbed onto a small localised volume ca 5 mm³, of the implant of dimensions 8×8×4 mm, (approximately 250 mm³). This copper thus represented 88 ng per cm³.

### Example 2

Brushite and hydroxyapatite materials were implanted in animals for 15 days in order to observe tissue ingrowth, specifically angiogenesis and vascularization. In order to facilitate observation of tissue ingrowth the implants were made in two mirror image halves that keyed into one another and split the pore cavity along its symmetrical axis (FIG. 2). A 5 μl solution of copper sulfate at low concentration (0.07 mM) was deposited at the end of the closed pore, dried, and implanted in the abdominal cavity (i.e., intraperitoneally) in mice for 15 days. The retrieved implants were opened and examined for tissue ingrowth (FIG. 2C). A new tissue with obvious microvessels (or capillaries) was formed particularly at the large open area, the crossing Y pore and the closed end pore. This is in comparison to control implants with no adsorption of copper (FIG. 2D). Microscopic observation demonstrated the formation of these microvessels appearing in the newly formed wound tissue (FIG. 2Cm) compared to control implants (FIG. 2Dm).

### Example 3

After an implantation of 15 day duration, higher doses of copper (10 times greater than example 2) adsorbed onto the materials at the end of the closed pore resulted in a vascular tissue.

### Example 4

Repeating the example 2 with 100 times more concentrated copper solution resulted in a pore infiltration by a tissue rich in leukocytes and dead cells. No blood vessels were observed.

### Example 5

Similar to the implants with copper as described above, solutions (3 μl per half implant) of manganese chloride, sodium selenium, silver nitrate, zinc chloride, and cobalt chloride diluted in Hank's balanced salt solution (vehicle) were adsorbed on the closed pore on each half. They were used respectively at 70 μM. The final amounts of the metals ranged from 10 to 200 ng per implant. These conditions were compared to vehicle (HBBS) as control and copper (70 μM)

After 15 days of implantation in peritoneal site in mice, the implants were retrieved and histological sections of the tissue ingrowth in the pores were processed. In the presence of manganese, a bloody tissue was formed in the pores (blind and open) and the presence of a clot with blood cells was confirmed on histological sections Similar observations were found with zinc and silver-loaded implant and the control implant with no orientation towards the closed pore (loaded pore). In the cobalt-loaded implant, the wound tissue was mildly inflammatory, and by histology, microvessels with transgression of inflammatory cells were observed (FIG. 4).

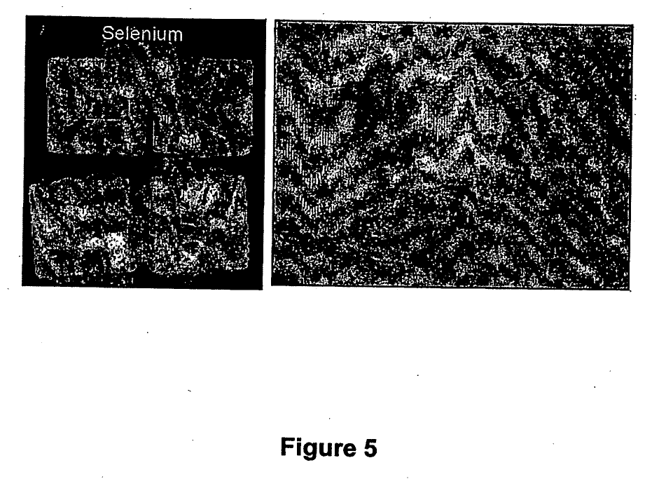

In the selenium-loaded implant, the tissue ingrowth was oriented towards the closed pore and histological sections showed an immature wound tissue (FIG. 5). This represents a selenium ion concentration of 33 ng per implant.

In conclusion, it is possible to stimulate wound healing by using selenium (at 0.25 ng/mm³ of the implant) and cobalt (at 0.45 ng per mm³ of the implant). Although there is a mild inflammatory response, cobalt enhances particularly blood vessel formation and be comparable to copper response whereas selenium may enhance tissue maturation.

Without wishing to be bound by theory, we believe that the_implant of the present invention releases ions transiently or in a pulsed fashion over a period of time, but not permanently and so might stop or diminish to non biologically active levels once the ‘tissue growth’ had occurred or, in the case of a degradable or soluble implant, simply would not be there anymore. Metal implants release ions as an undesired by product of the corrosion process for years. A burst release can occur shortly after implantation due to initial passivation. Many ions released from metal implants start life as wear particles, which are then phagocytosed.

In order to have the same release profile in different materials as in these example, various concentrations may be required depending on the form of the metal/non-metal, implant matrix, implantation site and the like.

### Example 6

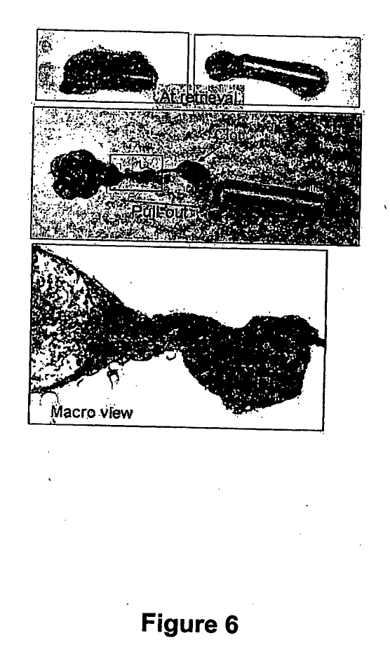

The main objective of this study was to cover the interior of a metallic tube (18 G needle) with copper for further application to induce angiogenesis and vascularisation into metal implants as used in dentistry and orthopedics. Using electroplating technology, a thin layer of copper was deposited in the internal surface of 8 mm sections cut from stainless steel needles. The outer surface was masked with tape and electroplating was performed using a 1.5 V AAA battery in a 10 μM copper sulfate solution with an electrode spacing of 5 cm for a duration of 3 minutes. After sterilization by heat, cylinders were implanted in peritoneal and subcutaneous sites. Control tubes (non-coated) and copper-coated tubes were compared after 15 days of implantation. At implant retrieval, control tubes were slightly integrated into the surrounding fatty tissue. The wound tissue found in the interior of the tube was very limited in the subcutaneous implant. In the peritoneal control implant, tissue filled with blood was found (FIG. 6). Observation under microscope showed no microvessel existing in the tissue pulled out from the tube.

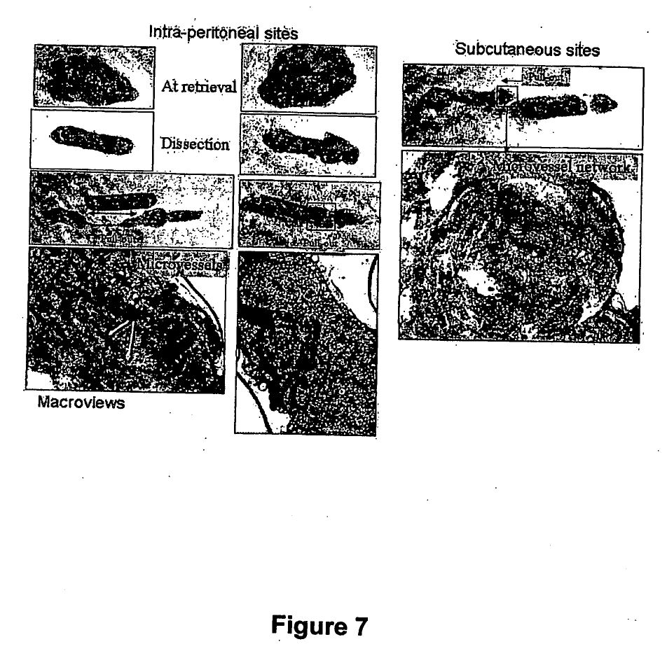

In the interior of the copper-coated tubes, a relatively extended wound tissue was more specifically observed in the peritoneal implants (FIG. 7) compared to the subcutaneous implant. However, the observation under microscope of the pulled out tissues showed obvious microvessels in the subcutaneous and peritoneal implants.

In conclusion, the host tissue that surrounded the metallic tubes during the implantation period migrated into the interior of the tube. However, microvessels and probably new capillaries are preserved in the presence of copper. Conversely, in the absence of copper no microvessels were observed.

### REFERENCES

Hölzel T., Rapid prototyping of phosphate of calcium structures by means of a reaction by the hardening process of cement. Diploma Thesis, University of Applied Sciences, Erlangen-Nürnberg, Germany, Aug. 19, 2005.
Barbucci R, Lamponi S, Magnani A, Piras F M, Rossi A, Weber E. Role of the Hyal-Cu (II) complex on bovine aortic and lymphatic endothelial cells behavior on microstructured surfaces. Biomacromolecules. 2005; 6:212-9.
Giavaresi G, Torricelli P, Formasari P M, Giardino R, Barbucci R, Leone G. Blood vessel formation after soft-tissue implantation of hyaluronan-based hydrogel supplemented with copper ions. Biomaterials. 2005; 26:3001-8.
Barbucci R; Rappuoli R (Assignee: Farmila-Thea Farmaceutici S.p.A. (Settimo Milanese, IT) Cross-linked hyaluronic acids and medical uses thereof. PCT PUB. NO.: WO00/27887; PCT PUB. Date: May 18, 2000
Rajalingam D, Kumar T K, Yu C. The C2A Domain of Synaptotagmin Exhibits a High Binding Affinity for Copper: Implications in the Formation of the Multiprotein FGF Release Complex. Biochemistry. 2005; 44:14431-42.
Sen C K, Khanna S, Venojarvi M, Trikha P, Ellison E C, Hunt T K, Roy S. Copper-induced vascular endothelial growth factor expression and wound healing. Am J Physiol Heart Circ Physiol. 2002; 282:H1821-7.
Goodman V L, Brewer G J, Merajver S D. Control of copper status for cancer therapy. Curr Cancer Drug Targets. 2005; 5:543-9.
Grzenkowicz-Wydra J, Cisowski J, Nakonieczna J, Zarebski A, Udilova N, Nohl H, Jozkowicz A, Podhajska A, Dulak J. Gene transfer of CuZn superoxide dismutase enhances the synthesis of vascular endothelial growth factor. Mol Cell Biochem. 2004; 264:169-81.
Atsumi K, Saito T, Komori M, Assignee: Kabushiki Kaisha Sangi (Tokyo, JP) Process for producing an antibacterial ceramic material U.S. Pat. No. 5,151,122; Sep. 29, 1992.
Sakuma S, Atsumi K, Fujita K; Assignee: Kabushiki Kaisha Sangi (Tokyo, JP) Anti-adhesion agent to water borne organisms U.S. Pat. No. 5,324,525, Jun. 28, 1994.
Saltman P D, Smith K T; Assignee: The Procter & Gamble Company (Cincinnati, Ohio); Regents of the University of California (Oakland, Calif.). Calcium and trace mineral supplements. U.S. Pat. No. 5,151,274; Sep. 29, 1992
Kweon Y J; Assignee: Samwoo Far Infra-Red Ray Co., Ltd. (KR Disinfectant ceramic composition. U.S. Pat. No. 5,187,124; Feb. 16, 1993
Zeng W, Arata M, Banba T; Assignee: Sun Medical Co., Ltd. (Modyama, JP) Primer solution composition for dental bonding U.S. Pat. No. 5,670,559; Sep. 23, 1997
Sakuma S, Atsumi K; Assignee: Sangi Co., Ltd. (Tokyo, JP) Dentifrice containing antibacterial material U.S. Pat. No. 5,468,489; Nov. 21, 1995
Hu, G. Copper stimulates proliferation of human endothelial cells under culture. J. Cell. Biochem. 1998; 69: 326-335.

All publications mentioned in this specification are hereby incorporated by reference.

### Other Embodiments

From the foregoing description, it will be apparent to one of ordinary skill in the art that variations and modifications may be made to the invention described herein to adapt it to various usages and conditions. Such embodiments are also within the scope of the present invention._These modifications are within the scope of this invention as defined in the appended claims:

1-103. (canceled)

104. An implant to stimulate tissue growth comprising:
a) a body having a body core and a body surface, and wherein the body is made of a material selected from the group consisting of one or more of a non-hydrogel polymer material, a ceramic material; and a metallic material; and
b) a tissue growth stimulating material being disposed within the body core or located on the body surface in an amount that is sufficient to stimulate tissue growth within the body core or adjacent to the body surface.

105. The implant of claim 104, wherein the tissue growth stimulating material is a metallic material and the metallic material is selected from the group consisting of an elemental metal and a metal ion.

106. The implant of claim 105, wherein the elemental metal is selected from the group consisting of copper and cobalt.

107. The implant of claim 105, wherein the metal ion is selected from the group consisting of a metal salt and a metal complex

108. The implant of claim 104, wherein the tissue growth stimulating material is a non-metallic material and the non-metallic material is selected from the group consisting of elemental selenium and selenium salt.

109. The implant of claim 104, further comprising a component in the body selected from the group consisting of a growth factor, cytokine and tissue inductive substance

110. The implant of claim 106, wherein the copper has a concentration of less than 20 μg per mm³ of implant material.

111. The implant of claim 104, in which the ceramic material is selected from the group consisting of bioceramic and bioglass.

112. The implant of claim 111, wherein the bioceramic material is selected from the group consisting of brushite and hydroxyapatite.

113. The implant of claim 104, wherein the amount of the tissue growth stimulating material is sufficient to produce an action selected from the group consisting of to stimulate angiogenesis, to promote vascularization, and to promote microvessel formation.

114. The implant of claim 104, in which the non-hydrogel polymer material is selected from the group consisting of a synthetic non-hydrogel polymer and a natural non-hydrogel polymer.

115. The implant of claim 104, wherein the body has pores.

116. The implant of claim 115, wherein the pores are approximately 100 μm and larger.

117. An implant to stimulate tissue growth comprising
a) a body having a body core and a body surface, the body being made from a hydrogel polymer material;
b) a tissue growth stimulating material being disposed within the body core or located on the body surface in an amount which is sufficient to stimulate tissue growth within the body core or adjacent to the body surface, and
c) wherein the tissue growth stimulating material is selected from the group consisting of one or more salts of selenium, cobalt, iron, zinc, magnesium, and manganese.

118. A method of stimulating tissue growth comprising:
a) attaching an element or a compound with the element, wherein the element is selected from the group consisting of copper, cobalt, selenium, iron, zinc, magnesium, and manganese, onto a biocompatible material to produce a coated biocompatible material,
b) placing the coated biocompatible material in a location in need of tissue stimulation,
c) stimulating the growth of a tissue, and
d) allowing the tissue to grow within and upon the coated biocompatible material.

119. The method of claim 118, wherein the element or compound is in a form selected from the group consisting of an elemental metal, salt, complex, ion, and chelate.

120. The method of claim 118, wherein the tissue is selected from the group of bone, skin, muscle, cartilage, connective, adipose, tendon and ligament.

121. The method of claim 118, wherein the copper has a concentration of less than 20 μg per mm³.
